(19) United States
(12) Patent Application Publication
(10) Pub. No.: US 2013/0116472 A1
(43) Pub. Date: May 9, 2013

(54) PROCESS FOR PREPARING UREA

(71) Applicant: Norbert Auner, Glashuetten (DE)

(72) Inventor: Norbert Auner, Glashuetten (DE)

(73) Assignee: SPAWNT PRIVATE S.A.R.L., Luxembourg (LU)

(21) Appl. No.: 13/580,006

(22) PCT Filed: Feb. 28, 2011
(86) PCT No.: PCT/EP11/52931

§ 371 Date: Dec. 5, 2012

(30) Foreign Application Priority Data
Feb. 26, 2010 (DE) 10 2010 009502.8

(51) Int. Cl. C07C 273/10

(52) U.S. Cl. CPC C07C 273/10
USPC 564/69

(57) ABSTRACT

A method of producing urea includes reacting SiO₂/Al₂O₃ or SiO₂/Al₂O₃-containing material, with addition of a carbon source, with gaseous nitrogen at elevated temperature to produce silicon nitride (Si₃N₄)/aluminum nitride (AlN) or silicon nitride/aluminum nitride-containing material; reacting the silicon nitride/aluminum nitride or silicon nitride/aluminum nitride-containing material in the presence of a basic alkali metal compound and/or alkaline-earth metal compound, with water at elevated temperature, to produce ammonia and alkali metal silicates/aluminates and/or alkaline earth metal silicates/aluminates; and reacting the ammonia with carbon dioxide to produce the urea.

### RELATED APPLICATIONS

This is a §371 of International Application No. PCT/EP2011/052931, with an international filing date of Feb. 28, 2011 (WO 2011/104387 A1, published Sep. 1, 2011), which is based on German Patent Application No. 10 2010 009502.8, filed Feb. 26, 2010, the subject matter of which is incorporated by reference.

### TECHNICAL FIELD

This disclosure relates to a method for producing urea by reacting ammonia with carbon dioxide.

### BACKGROUND

Methods of this type for the industrial production of urea are common. To produce the ammonia required for these methods, there are a multiplicity of processes, of which the Haber-Bosch process is the best-known. Also known is the process called the “Serpek” process, which relates to hydrolysis of nitrides (2AlN+3H₂O→4Al₂O₃+2NH₃). One of the most important nitrides is silicon nitride (Si₃N₄). Preparation of silicon nitride from SiO₂ sources by carbonitriding is known. In carbonitriding, silicon dioxide is reacted at elevated temperature with gaseous nitrogen by addition of a carbon source.

DE 10 2009 011 311.8, which was not published before the priority date of this application, describes a method for producing ammonia by reacting SiO₂ or SiO₂-containing material, with addition of a carbon source, with gaseous nitrogen at elevated temperature to give silicon nitride (Si₃N₄) or material containing silicon nitride, and reacting the resultant silicon nitride or material containing silicon, in the presence of a basic alkali metal compound and/or alkaline earth metal compound, with water at elevated temperature to give ammonia and alkali metal silicates and/or alkaline earth metal silicates.

It could, however, be helpful to provide a method for producing urea that is easy to carry out and that allows particularly effective utilization of natural resources.

### SUMMARY

I provide a method of producing area including reacting SiO₂/Al₂O₃ or SiO₂/Al₂O₃-containing material, with addition of a carbon source, with gaseous nitrogen at elevated temperature to produce silicon nitride (Si₃N₄)/aluminum nitride (AlN) or silicon nitride/aluminum nitride-containing material; reacting the silicon nitride/aluminum nitride or silicon nitride/aluminum nitride-containing material in the presence of a basic alkali metal compound and/or alkaline-earth metal compound, with water at elevated temperature, to produce ammonia and alkali metal silicates/aluminates and/or alkaline earth metal silicates/aluminates; reacting the ammonia with carbon dioxide to produce the urea.

### DETAILED DESCRIPTION

We provide a method comprising the reaction of SiO₂/Al₂O₃ or of SiO₂/Al₂O₃-containing material, with addition of a carbon source, with gaseous nitrogen at elevated temperature to give silicon nitride (Si₃N₄)/aluminum nitride (AlN) or silicon nitride/aluminum nitride-containing material, and reaction of the resultant silicon nitride/aluminum nitride or silicon nitride/aluminum nitride-containing material, in the presence of a basic alkali metal compound and/or alkaline-earth metal compound, with water at elevated temperature to give ammonia and alkali metal silicates/aluminates and/or alkaline earth metal silicates/aluminates, the ammonia obtained being reacted with carbon dioxide to give urea.

The method thus makes use of Si and/or Al as a carrier material for production of ammonia, which is reacted, conventionally, with carbon dioxide to give urea. The method circumvents the Haber-Bosch process for preparation of ammonia, thereby removing the need to use elevated pressures.

The method is a three-stage method in which, in a first stage, silicon nitride and/or aluminum nitride is prepared and, in a second stage, ammonia is prepared from the silicon nitride and/or aluminum nitride. In a third stage, the ammonia is reacted with CO₂ to give urea. Reaction of the silicon nitride/aluminum nitride or silicon nitride/aluminum nitride-containing material takes place in the presence of a basic alkali metal compound and/or alkaline earth metal compound with water. Owing to the fact that not only the substances required to obtain silicon nitride/aluminum nitride (SiO₂ or SiO₂-containing material and/or Al₂O₃ or Al₂O₃-containing material, carbon source gaseous nitrogen), but also the substances needed to give ammonia (basic alkali metal compound and/or alkaline earth metal compound, water) and CO₂ for the reaction to give urea are available as natural, cheap resources, the method can be implemented easily and cost-effectively. Since, moreover, the method does not require elevated pressures, but instead merely elevated temperatures, the method can also be carried out relatively simply and inexpensively from the standpoint of process engineering.

A suitable starting point for the method is SiO₂ or SiO₂-containing material, more particularly in the form of sand (quartz sand), silicates, aluminosilicates, clays and the like, and also Al₂O₃ or Al₂O₃-containing material such as bauxite or the like. It is not necessary to use pure starting material. Instead, the material may also have corresponding impurities or additions, provided it is SiO₂-containing and/or silicate-containing and/or Al₂O₃-containing and/or aluminate-containing. There is, therefore, no need for costly and inconvenient purification measures.

As carbon-containing materials (carbon sources) it is possible to use substances such as, for example, bituminous coals, brown coals, coke or else coals from regenerative sources such as, for example, charcoal, activated carbon or coals obtainable by carbonization of agricultural byproducts such as straw, maize straw, rape straw, and rice straw. Preferred carbon-containing materials are those obtained from thermolysis of regeneratively obtained carbon-containing materials in the absence of oxygen or with limited oxygen supply. Nonlimiting examples of such carbon-containing materials are biomass such as wood, straw, reed and the like.

A further advantage of the method is that there is no need to prepare pure silicon nitride and/or aluminum nitride. Instead, to produce ammonia, it is sufficient to generate silicon nitride and/or aluminum nitride-containing material, and so, as mentioned, there is no need for costly and inconvenient measures to purify the starting material or materials. It is therefore possible to make use, as inexpensive starting materials, of, for example, bauxite (Al(OH)₃/AlO(OH)), still containing significant quantities of SiO₂, Fe₂O₃ and the like, or quartz sand, which may still contain feldspars, limestone (CaCO₃), gypsum, sulfides and the like, for production.

It is essential that the reaction of the resulting silicon nitride/aluminum nitride or silicon nitride/aluminum nitride-containing material with water (steam) takes place in the presence of a basic alkali metal compound and/or alkaline earth metal compound. This basic alkali metal compound and/or alkaline earth metal compound may be added to the silicon nitride/aluminum nitride or silicon nitride/aluminum nitride-containing material before addition of water. As a source thereof it is also possible to add a compound of this kind which releases a basic alkali metal compound and/or alkaline earth metal compound at the corresponding operational temperature. Nonlimiting examples of this are oxides, sulfides, and hydroxides of the alkali metals/alkaline earth metals, and alkali metal silicates/carbonates. In each case, the reaction with water must take place in a basic environment.

An SiO₂/Al₂O₃-containing material may be used which already comprises a basic alkali metal compound and/or alkaline earth metal compound or a source thereof. In this instance, therefore, no basic alkali metal compound and/or alkaline earth metal compound or a source thereof is added, but, instead, the starting material used already comprises such a compound or a source thereof. This may be realized, for example, through the use of a material containing SiO₂/Al₂O₃ that comprises constituents or impurities which release a basic alkali metal compound and/or alkaline earth metal compound at the corresponding operational temperature. Nonlimiting examples of this are feldspars, lime, dolomite, gypsum or sulfides, nitrates sulfates of the alkali metals/alkaline earth metals.

In addition to SiO₂/Al₂O₃ or SiO₂/Al₂O₃-containing material as starting material, a basic alkali metal compound and/or alkaline earth metal compound or a source thereof may be used from the start. Therefore, a starting material mixture may be used which comprises not only SiO₂/Al₂O₃ or SiO₂/A₂O₃-containing material, but also a basic alkali metal compound and/or alkaline earth metal compound or a source thereof. In this case as well, the source of the basic alkali metal compound and/or alkaline earth metal compound then liberates the basic alkali metal compound and/or alkaline earth metal compound at the corresponding operational temperature.

A key advantage of the method is that it can be carried out as a circulation operation. In that case, the alkali metal silicates/aluminates and/or alkaline earth metal silicates/aluminates obtained as end product are used again as starting product, i.e., as SiO₂/Al₂O₃-containing material. Depending on whether the alkali metal silicates/aluminates and/or alkaline earth metal silicates/aluminates obtained still comprise a source of a basic alkali metal compound and/or alkaline earth metal compound, it is then no longer necessary to add a new basic alkali metal compound and/or alkaline earth metal compound or a corresponding source thereof. It is clear that this aspect has the advantage that the alkali metal silicate/aluminate material and/or alkaline earth metal silicate/aluminate material obtained in the production of ammonia can be used specifically again as a starting product, thereby allowing particularly effective utilization of the products used for the method. The required SiO₂/Al₂O₃ or SiO₂/Al₂O₃-containing material must therefore merely be supplemented. Therefore, ammonia is obtained from SiO₂/Al₂O₃ or from SiO₂/AlO₃-containing material in a circulation operation.

Oxides, hydroxides, silicates and/or carbonates are used preferably as basic alkali metal compound and/or alkaline earth metal compound. As a source of such a compound it is, therefore, preferred to use one which releases corresponding oxides, hydroxides, silicates and/or carbonates. Nonlimiting examples of this are feldspars such as albite or orthoclase, lime, dolomite, gypsum, sodium carbonate, soda or sulfides, nitrates, and sulfates of the alkali metals/alkaline earth metals, e.g., NaNO₃ Na₂S, K₂SO₄.

As already mentioned, both steps of the method use elevated temperatures, and it is necessary, accordingly, for thermal energy to be supplied. This may take place in a conventional way. Particularly preferably, the elevated temperature in the first and/or second method step, however, is generated by microwave energy. This represents a particularly effective way of achieving the corresponding reaction temperatures to obtain the required reactive form of N₂ in the first step, more particularly by light arcs on the C center. In the third step as well it is possible to use microwave energy to achieve the corresponding temperatures selected.

More particularly, the reaction to give silicon nitride/aluminum nitride or silicon nitride/aluminum nitride-containing material is carried out preferably at a temperature of 1100-2000° C., more preferably 1250-1500° C. The reaction to give ammonia is carried out preferably at a temperature of 200-1000° C., preferably 400-800° C.

Reference has already been made above to the fact that, when the starting material used for the thermal preparation of nitride already comprises one or more sources of basic alkali metal compounds and/or alkaline earth metal compounds, more particularly alkali metal oxides/alkaline earth metal oxides, the nitride obtained is already enriched with basic material, and so it may be possible not to carry out further addition of basic material. A reaction with steam at elevated temperatures is then sufficient for the release of ammonia.

The product of the ammonia synthesis, i.e., the resultant alkali metal silicates/aluminates and/or alkaline earth metal silicates/aluminates, may, following addition of further carbon, be suitable directly again for formation of nitride, provided this product still comprises corresponding basic material. Further addition of basic material is superfluous in that event.

Starting materials containing silicon dioxide include those which comprise aluminum such as aluminosilicates and argillaceous earths. Nitride preparation in that case results in silicon nitride, with aluminum nitride as an impurity. The silicon nitride obtained may also be present, for example, in the form silicon oxynitride.

Starting materials preferably, in addition to SiO₂ in the form of sand, more particularly quartz sand, and Al₂O₃ (bauxite), include materials comprising alkali metal silicates and/or alkaline earth metal silicates, and also aluminosilicates. These materials have the advantage that they can automatically provide the basic alkali metal compounds and/or alkaline earth metal compounds (oxides, hydroxides and the like) for the operation without any need for these materials to be added later. With regard to the starting materials used, therefore, it is possible to do without, for example, extensive purification measures since silicate-containing materials of this kind are desired as starting material and it is not absolutely necessary to use pure SiO₂ or Al₂O₃.

Particularly preferably, the carbon source is obtained by pyrolysis of biomass (wood, straw, rice straw, reed, foliage, prunings, sawing chips and the like). Such pyrolysis yields hydrogen (H₂), carbon monoxide (CO₂), and more or less pure carbon (in the form of charcoal, carbonized material and the like), which is used as carbon source to obtain silicon nitride/aluminum nitride. The carbon monoxide (CO) generated in the course of the pyrolysis is preferably converted into carbon dioxide (CO₂) which is reacted with the ammonia produced, to give urea.

Pyrolysis temperatures used are preferably temperatures of ≧800° C.

The biomass is usefully dried before pyrolysis. This is normally necessary in view of the fluctuating water contents of the biomass.

Pyrolysis is preferably carried out without addition of steam to obtain the maximum amount of carbon.

Hydrogen and carbon monoxide substances produced in the pyrolysis are obtained in the form of synthesis gas (a mixture of H₂ and CO), which is preferably burned for energy recovery, with the resultant CO₂ being reacted with the ammonia to give urea.

Therefore, urea can be obtained from biomass, in other words from a renewable material, meaning that there is no need for fossil fuels for the method. The synthesis gas obtained in the pyrolysis of the biomass is used to generate energy, with the CO₂ produced in the process being largely used to obtain urea and, hence, not released to a great extent into the atmosphere. The Si and/or Al substances required as nitrogen carriers are available to a large extent in particular in the form of SiO₂ or silicate-containing materials, and can in any case, as outlined above, be recovered or recycled from the end products generated during the production of ammonia. Overall, therefore, the method can be carried out simply and inexpensively, and utilizes natural resources without consuming fossil fuels.

The terms “silicon nitride/aluminum nitride” and “silicon nitride/aluminum-nitride-containing material” used herein signify that the substances in question relate to “silicon nitride and/or aluminum nitride” and “silicon nitride and/or aluminum nitride-containing material.”

### WORKING EXAMPLES

### 1. Thermal Syntheses of Silicon Nitride

In preparation for the experiment, powdered activated carbon, silica gel 60 (particle size <0.063 mm) from Merck, and starch from the company Classic are mixed in a ratio of 2:1:1 and slurried with water to give a highly viscous liquid. The mixture is squirted in small strips onto a metal sheet and dried in a drying cabinet at 150° C. The solid obtained is comminuted into granules with a size of approximately 0.5 cm³. 5-6 g of these granules are introduced as plugs between quartz wool into a quartz tube having a diameter of 25 mm, which is clamped vertically in a tube oven from Gero and is supplied with nitrogen gas from below. The sample is calcined in a stream of nitrogen at 0.5 l/min for an hour at 650° C. and for half an hour at 750° C. The temperature is subsequently raised to the desired reaction temperature, and the material is heated for 3 hours. It is cooled to room temperature, still in a stream of nitrogen, and finally, in a stream of air at 4 l/min, the remaining activated carbon is burnt at 650° C. for 3 hours.

### 1a) Reaction of Silicon Dioxide with Activated Carbon and Nitrogen in a Tube Oven, at Temperatures of 1330° C. and 1380° C.

Starting materials: granules of powdered activated carbon, silica gel 60, and starch (2:1:1) measuring 0.5 cm³-1 cm³
Products: cristobalite, silicon nitride, silicon oxynitride
Reaction time: 3 h
Nitrogen stream: 0.5 l/min

The solid products obtained were analyzed by X-ray powder diffractometry.

### 1b) Reaction of Silicon Dioxide with Activated Carbon and Nitrogen in a Tube Oven, at Temperatures of 1430° C. and 1480° C.

Starting materials: granules of powdered activated carbon, silica gel 60, and starch (2:1:1) measuring 0.5 cm³-1 cm³
Products: silicon nitride
Reaction time: 3 h
Nitrogen stream: 0.5 l/min

The solid products obtained were analyzed by X-ray powder diffractometry.

### 2. Microwave-Assisted Syntheses of Silicon Nitride

The preparation of silicon nitride differs from the thermal synthesis largely only in the batch size. 2-3 g of the granules produced, comprising a mixture of powdered activated carbon, silica gel 60, and starch, are calcined, as plugs between quartz wool in a quartz tube having a diameter of 25 mm, under reduced pressure (10⁻³ mbar) of 100 W until the pressure remains constant. The power is slowly raised at the end, and again the presence of a constant pressure is awaited. By fine tuning, the system is now supplied with nitrogen up to the desired pressure. When the nitrogen pressure is regulated so that a violet-colored plasma is maintained in the reaction zone, silicon nitride is the main product of the reaction. The reactions are carried out for three hours, followed by cooling in a stream of nitrogen at ambient pressure. The remaining activated carbon is burnt off in a stream of air at 4 l/min by means of a Heraeus tube oven at 650-700° C. In the case of reactions in a stream of nitrogen under atmospheric pressure, the granules are calcined beforehand in the Gero tube oven.

2a) Reaction of silicon oxide with activated carbon and nitrogen in a monomode microwave, power 750 W

Starting materials: granules of powdered activated carbon, silica gel 60, and starch (2:1:1) measuring 0.5 cm³-1 cm³
Products: cristobalite, quartz, silicon carbide, silicon nitride
Reaction time: 3 h
Nitrogen pressure: 200 mbar

The solid products obtained were analyzed by X-ray powder diffractometry.

### 2b) Reaction of Silicon Oxide with Activated Carbon and Nitrogen in a Monomode Microwave, Power 600 W, 750 W or 900 W, Violet Plasma

Starting materials: granules of powdered activated carbon, silica gel 60, and starch (2:1:1) measuring 0.5 cm³-1 cm³
Products: cristobalite, silicon carbide, silicon nitride (main product) Reaction time: 3 h
The solid products obtained were analyzed by X-ray powder diffractometry.

### 3. Thermal synthesis of Aluminum Nitride

For the preparation of aluminum nitride, powdered activated carbon, granulated activated carbon (granule size 1.5 mm), aluminum oxide (particle size 50 to 200 gm) from Merck, and starch from the company Classic are mixed in a ratio of 2:2:1:1 and the silicon nitride synthesis procedure is carried out in the same way as for the sample preparation of the starting materials. 5.3 g of these granules are calcined in a stream of nitrogen at 0.5 l/min for an hour at 650° C. and for a further half an hour at 750° C. The temperature is subsequently raised to the reaction temperature. When the reaction is at an end, cooling takes place in a stream of nitrogen and the product residual activated carbon is burnt in a dried (with potassium hydroxide and molecular sieve) stream of air at 650° C. for 3 hours.

3a) Reaction of Aluminum Oxide with Activated Carbon and Nitrogen in a Tube Oven, Temperature 1430° C.

Starting materials: granules of powdered activated carbon, granulated activated carbon, aluminum oxide, and starch (2:2:1:1) measuring 0.5 cm³-1 cm³
Products: aluminum nitride
Reaction time: 3 h
Nitrogen stream: 0.5 l/min

The solid products obtained were analyzed by X-ray powder diffractometry.

### 4. Microwave-Assisted Synthesis of Aluminum Nitride

1.5-2.5 g of the granules of aluminum oxide, activated carbon, and starch are heated under reduced pressure (10⁻³ mbar) at 100 W until the pressure remains constant. The power is raised to the desired level, and again the establishment of the constant pressure is awaited. Fine tuning is then carried out to supply nitrogen to the selected pressure. The reactions are carried out for 3 hours, followed by cooling to room temperature in a stream of nitrogen and by freeing from residual carbon in a dried stream of air at 4 l/min at 650-700° C.

### 4a) Reaction of Aluminum Oxide with Activated Carbon and Nitrogen in a Monomode Microwave, Power 600 W

Starting materials: granules of powdered activated carbon, granulated activated carbon, aluminum oxide, and starch (2:2:1:1) measuring 0.5 cm³-1 cm³
Products: aluminum nitride, aluminum oxynitride
Reaction time: 3 h
Nitrogen pressure: 300 mbar
The solid products obtained were analyzed by X-ray powder diffractometry.

### 4b) Reaction of Aluminum Oxide with Activated Carbon and Nitrogen in a Monomode Microwave, Power 800 W, Violet Plasma

Starting materials: granules of powdered activated carbon, granulated activated carbon, aluminum oxide, and starch (2:2:1:1) measuring 0.5 cm³-1 cm³
Products: aluminum nitride
Reaction time: 3 h

The solid products obtained were analyzed by X-ray powder diffractometry.

An analogous procedure can also be carried out with silicon oxide/aluminum oxide mixtures.

### 5. Hydrolysis of Silicon Nitride

The hydrolysis of commercial, thermally prepared silicon nitride takes place here by way of example in a tube oven at 700° C. 0.5 g of silicon nitride from Aldrich Chemical Company (−325 mesh), with a slight stoichiometric excess of the bases, is introduced as plugs between quartz wool into a quartz tube. Water is introduced dropwise by means of a syringe (operated by a syringe motor) at 7.6 ml/h into a T-piece made of Duran glass. The T-piece and the quartz tube are wrapped with a heating belt, which carries out preheating at a temperature of 100-200° C. and thus evaporates the water. Nitrogen is supplied by the second connection of the T-piece to transport the steam through the reaction space. The ammonia formed is passed through two wash bottles, the latter of which being filled with 25 ml of 0.5 M sulfuric acid. By back-titration with 1 M sodium hydroxide solution it is possible, finally, to determine the ammonia yield. For the purpose of comparison, 0.3 g of selected products of microwave syntheses is hydrolyzed with 1.0 g of sodium carbonate in the same experimental setup and with the same experimental procedure.

### 5a) Reaction of Silicon Nitride (Commercial) with Sodium Carbonate and Steam

Starting materials: silicon nitride (commercial), sodium carbonate, steam
Products: ammonia, sodium silicate, silicon nitride
NH3 yield: 80.9% for 5 h reaction time or 75.1% for 2.5 h reaction time
Tube diameter: 16 mm

### Temperature: 700° C.

Water addition: 7.6 ml/min

The solid products obtained were analyzed by X-ray powder diffractometry.

### 5b) Reaction of Silicon Nitride (Commercial) with Calcium Carbonate and Steam

Starting materials: silicon nitride (commercial), calcium carbonate, steam
Products: ammonia, calcium silicate, calcium oxide, calcium hydroxide, silicon nitride
NH3 yield: 60.6%
Tube diameter: 16 mm

### Temperature: 700° C.

Reaction time: 5 h
Water addition: 7.6 ml/min

The solid products obtained were analyzed by X-ray powder diffractometry.

### 6. Hydrolysis of Aluminum Nitride

0.4-0.6 g of the aluminum nitride synthesized beforehand is admixed in a Schlenk flask with 20 ml of 20 percent strength sodium hydroxide solution, and heated under reflux for two hours. Nitrogen is fed in via the gas port of the Schlenk flask, and the ammonia produced is transferred into a wash bottle containing 25 ml of 0.5 M sulfuric acid. By back-titration with 1 M sodium hydroxide solution, the amount of ammonia produced is ascertained.

### 6a) Reaction of Aluminum Nitride with Steam

Starting materials: aluminum nitride
Products: ammonia (other products not determined)
NH3 yield: 85.3%
Reaction time: 2 h

### 7. Continuous Reaction of Quartz Sand to Give Ammonia

Quartz sand was reacted with addition of carbon and gaseous nitrogen at a temperature of 1300° C. to give silicon nitride. Following addition of Na₂CO₃, the silicon nitride obtained was reacted with steam at 800° C. to give ammonia. An 85% yield of NH₃ was achieved in this operation.

### 8. Synthesis of Urea From the Ammonia Released

The ammonia generated is reacted in excess with carbon dioxide at about 200° C. under a pressure of 250 bar to give a urea melt, which is cooled with depressurization and is processed further mostly in solid form to give fertilizers. Unreacted ammonia is returned to the operation.

1. A method of producing urea comprising:
reacting SiO₂/Al₂O₃ or SiO₂/Al₂O₃-containing material, with addition of a carbon source, with gaseous nitrogen at elevated temperature to produce silicon nitride (Si₃N₄)/aluminum nitride (AlN) or silicon nitride/aluminum nitride-containing material;
reacting the silicon nitride/aluminum nitride or silicon nitride/aluminum nitride-containing material in the presence of a basic alkali metal compound and/or alkaline-earth metal compound, with water at elevated temperature, to produce ammonia and alkali metal silicates/aluminates and/or alkaline earth metal silicates/aluminates; and
reacting the ammonia with carbon dioxide to produce the urea.

2. The method according to claim 1, wherein the basic alkali metal compound and/or alkaline earth metal compound or a source thereof is added to the silicon nitride/aluminum nitride or silicon nitride/aluminum nitride-containing material before the addition of the water.

3. The method according to claim 1, wherein the basic alkali metal compound and/or alkaline earth metal compound or a source thereof comprises an SiO₂- or Al₂O₃-containing material.

4. The method according to claim 1, further comprising in addition to SiO₂/Al₂O₃ or SiO₂/Al₂O₃-containing material as a starting material, reacting a basic alkali metal compound and/or alkaline earth metal compound or a source thereof with the gaseous nitrogen.

5. The method according to claim 1, wherein a basic alkali metal compound and/or alkaline earth metal compound is liberated from a corresponding source under operational conditions.

6. The method according to claim 1, carried out as a circulation operation and wherein the alkali metal silicates/aluminates and/or alkaline earth metal silicates/aluminates are reused as SiO₂/Al₂O₃-containing starting material.

7. The method according to claim 1, wherein oxides, hydroxides and/or carbonates are used or generated as the basic alkali metal compound and/or alkaline-earth metal compound.

8. The method according to claim 1, wherein the reaction to which produces the silicon nitride/aluminum nitride or silicon nitride/aluminum nitride-containing material is carried out at a temperature of 1100-2000° C.

9. The method according to claim 1, wherein the reaction to which produces the ammonia from the silicon nitride/aluminum nitride or silicon nitride/aluminum nitride-containing material is carried out at a temperature of 200-1000° C.

10. The method according to claim 1, wherein the elevated temperature in the first and/or second method step is generated by microwave energy.

11. The method according to claim 1, wherein the carbon source is obtained by pyrolysis of biomass.

12. The method according to claim 11, wherein the CO generated during the pyrolysis is converted into CO₂ and reacted with the ammonia to produce the urea.

13. The method according to claim 11, wherein the pyrolysis is carried out at temperatures ≧800° C.

14. The method according to claim 11, wherein the biomass is dried before the pyrolysis.

15. The method according to claim 11, wherein the pyrolysis is carried out without addition of steam.

16. The method according to claim 11, wherein synthesis gas (H₂, CO) obtained during the pyrolysis is burned to obtain energy and resulting CO₂ is reacted with the ammonia to produce the urea.

17. The method according to claim 12, wherein the pyrolysis is carried out at temperatures ≧800° C.
